(19) United States
(12) Patent Application Publication
(10) Pub. No.: US 2018/0185191 A1
(43) Pub. Date: Jul. 5, 2018

(54) Assessing Temperature Tolerance of Eyelids for Treating Meibomian Gland Dysfunction (MGD) and Dry Eye

(71) Applicant: Graham Stetson, Boston, MA (US)

(72) Inventor: Graham Stetson, Boston, MA (US)

(21) Appl. No.: 15/786,634

(22) Filed: Oct. 18, 2017

Related U.S. Application Data
(60) Provisional application No. 62/409,634, filed on Oct. 18, 2016.

(51) Int. Cl. A61F 7/08; A61B 5/01; A61B 5/00; A61F 7/00

(52) U.S. Cl. CPC A61F 7/08; A61B 5/01; A61B 5/4836; A61F 2007/0096; A61F 7/007; A61F 2007/0004; A61F 2007/0086; A61B 5/6821

(57) ABSTRACT

A method and apparatus for treating Meibomian Gland Dysfunction (MGD) and Dry Eye is presented. The device includes a heating element sized to cover an eye of a person, a cover disposed surrounding the heating element, a temperature sensor disposed on an outside surface of the cover; and a controller in electrical communication with the heating element and the temperature sensor, the controller for setting and maintaining a temperature of the heating element.

### BACKGROUND

The meibomain glands, present in the eyelids, produce the oily component of tears. The purpose of this oily layer is to limit evaporation of tears while the eyes are open. Since the vast majority of dry eye cases are due in some part to a deficiency of the layer of tears provided by the meibomian glands, this indicates some sort of meibomian gland dysfunction (MGD). In fact, MGD is now thought of as one of the chief causes of dry eye. MGD is a chronic, diffuse abnormality of the meibomian glands, commonly characterized by terminal duct obstruction and/or qualitative/quantitative changes in the glandular secretion. It may result in alteration of the tear film, symptoms of eye irritation, clinically apparent inflammation, and ocular surface disease. Current data on dry eye shows that 86% of cases are related, exclusively or in part, to excessive evaporation of tear film.

Dry eye is a multifactorial disease of the tears and ocular surface that results in symptoms of discomfort, visual disturbance, and tear film instability with potential damage to the ocular surface. It is accompanied by increased osmolarity of the tear film and inflammation of the ocular surface. Dry eye is a disorder of the tear film due to tear deficiency or excessive tear evaporation which causes damage to the interpalpebral ocular surface and is associated with symptoms of discomfort. Dry eye occurs when the eye does not produce tears properly, or when the tears are not of the correct consistency and evaporate too quickly. In addition, inflammation of the surface of the eye may occur along with dry eye.

It is believed that MGD may be the most common cause of evaporative dry eye and may also have some association with aqueous-deficient dry eye. Overview reports on dry eye have suggested meibomian oil deficiency as an intrinsic factor associated with the disease.

Warm compresses are commonly prescribed for MGD. The increasing prevalence of dry eye and the knowledge that MGD is its primary cause has led to a proliferation of commercial warm compresses, and this implies that there is an increasing market for the product, and that there are more people using warm compresses. Because there is no standard or safety information for warm compresses, they are not all effective, and they can present a hazard to the user.

Established dry eye experts have used compresses heated to primarily two different standards in their research, 45° C. and 47° C. The dry eye experts have also utilized two methods, heated water and heated oil, flowing over the skin to induce hyperthermia. It is easy to assume that the higher the temperature of the heated substance applied to the skin, the shorter the time needed to achieve a burn. However, it has been found that an inverse logarithmic relationship between temperature and time, that higher temperatures require disproportionally less time to burn; e.g. at 44° C. it takes 6 hours to produce epithelial damage, at 47° C. it takes 25 minutes, and at 60° C. it takes 5 seconds to create the same effects.

The composition, thickness, vascularization, and innervation of skin of the eyelid are different than that of the chest and underside of the forearms, which were the two areas of skin examined by prior researchers. Heat was delivered using a liquid media circulating against the skin at a constant temperature. This level of contact, and the use of a media with high heat capacity, generated maximal heat transfer to the skin.

The aim of warm compress therapy is to increase blood flow to eyelid tissues, or to bring Meibomian gland secretions to their melting point. As such, to provide maximum therapeutic potential, it is best to apply as much heat as is possible while maintaining comfort and patient safety. Research has indicated that temperatures higher than 47° C. could be utilized in warm compress therapy safely. At the very least, determining an upper limit of temperature tolerance will allow the establishment of standard temperatures for warm compress application.

Several assumptions can be made about thermal tolerance of the skin. Exposure to temperatures of 49° C. for at least 8 minutes will produce sub-threshold responses (i.e. no loss of or damage to epidermis). Exposure to temperatures of 51° C. for at least 2 minutes will produce sub-threshold responses. And exposure to temperatures of 53° C. for at least 30 seconds will produce sub-threshold responses. However, since these experiments were conducted on the chest and arms, it is unknown if these data apply directly to the eyelids. Another study looked at short-term immersion of cultured skin in a hot water bath, and found that exposure of thin layers of skin to temperatures above 58° C. resulted in a type of non-inflammatory programmed cell death.

### SUMMARY

Warm compresses are recommended by doctors for a number of conditions related to ocular health. These include, but are not limited to MGD and dry eye. However, in practice, there is no standardization in recommended temperatures. In part, this is due to the limited data available on patient safety and heat tolerance. A review of literature yields little data as to how the temperatures recommended for warm compresses were established.

In a particular embodiment a device for treating Meibomian Gland Dysfunction (MGD) and Dry Eye includes a heating element sized to cover an eye of a person. The device further includes a cover disposed surrounding the heating element. Additionally, the device features a temperature sensor disposed on an outside surface of an eyelid of the eye of the person. A controller in electrical communication with the heating element and the temperature sensor is also included, the controller for setting and maintaining a temperature of the heating element.

Note that each of the different features, techniques, configurations, etc. discussed in this disclosure can be executed independently or in combination. Accordingly, the present invention can be embodied and viewed in many different ways. Also, note that this summary section herein does not specify every embodiment and/or incrementally novel aspect of the present disclosure or claimed invention. Instead, this summary only provides a preliminary discussion of different embodiments and corresponding points of novelty over conventional techniques. For additional details, elements, and/or possible perspectives (permutations) of the invention, the reader is directed to the Detailed Description section and corresponding figures of the present disclosure as further discussed below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts an image of a heating element in accordance with embodiments of the invention.

FIG. 2 depicts an image of a temperature sensor in accordance with embodiments of the invention.

FIG. 3 depicts the controller in accordance with embodiments of the invention.

FIG. 4 depicts an image of two thermistors in accordance with embodiments of the invention.

FIG. 5 depicts a diagram showing placement of the thermistors and temperature sensor during testing of the device in accordance with embodiments of the invention.

FIGS. 6A and 6B are diagrams of a bilateral device for commercial use in accordance with embodiments of the invention.

### DETAILED DESCRIPTION

The embodiments set forth below represent the necessary information to enable those skilled in the art to practice the invention and illustrate the best mode of practicing embodiments of the invention. Upon reading the following description in light of the accompanying figures, those skilled in the art will understand the concepts of the invention and recognize applications of these concepts not particularly addressed herein. It should be understood that these concepts and applications fall within the scope of the disclosure and the accompanying claims.

The preferred embodiment of the invention will now be described with reference to the accompanying drawings. The invention may, however, be embodied in many different forms and should not be construed as limited to the embodiment set forth herein; rather, this embodiment is provided so that this disclosure will be thorough and complete, and will fully convey the scope of the invention to those skilled in the art. The terminology used in the detailed description of the particular embodiment illustrated in the accompanying drawings is not intended to be limiting of the invention. In the drawings, like numbers refer to like elements.

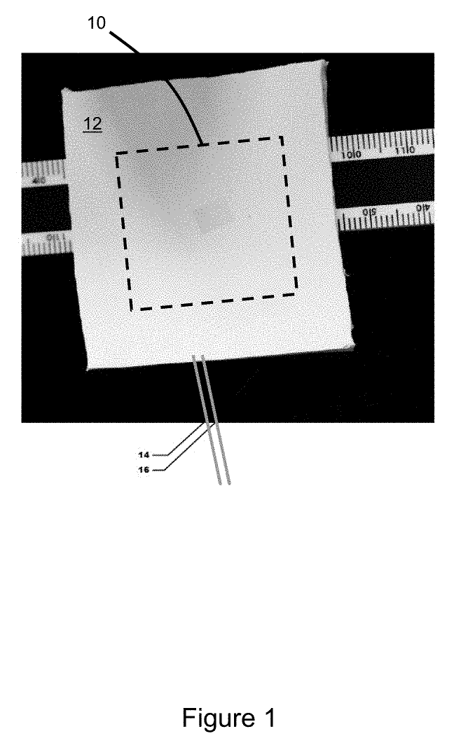

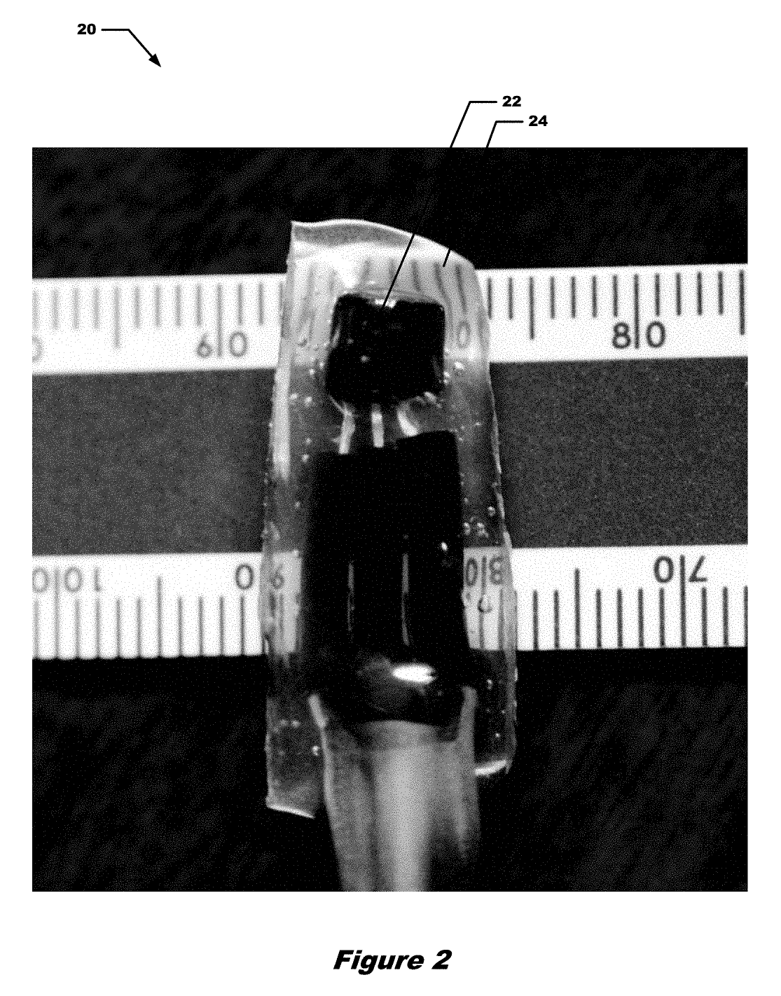

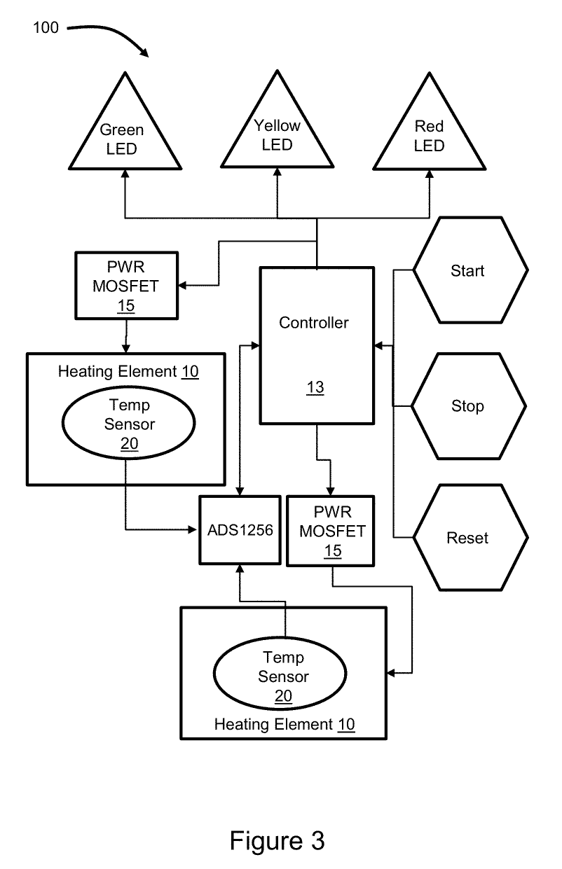

Referring to FIGS. 1 through 3, the warming apparatus includes a pair of 12V 24 W 30 mm×41 mm flexible Kapton-covered heating elements 10 enclosed in a waterproof, food grade, 1/16″ nitrile rubber casing. Leads 14 and 16 connect the heating element to a controller 13. Each heating element 10 is monitored by a temperature sensor element 20 comprising a Maxim DS18B20 (San Jose, Calif.) temperature sensor 22 enclosed in a waterproof urethane casing 24. The temperature sensor element 20 and the heating element 10 are controlled via the Arduino Uno microcontroller board, which will be programmed via the Arduino IDE using the C++ programming language. The heating element is activated through an IRF520 power MOSFET 15, allowing for varying intensities of heat generation.

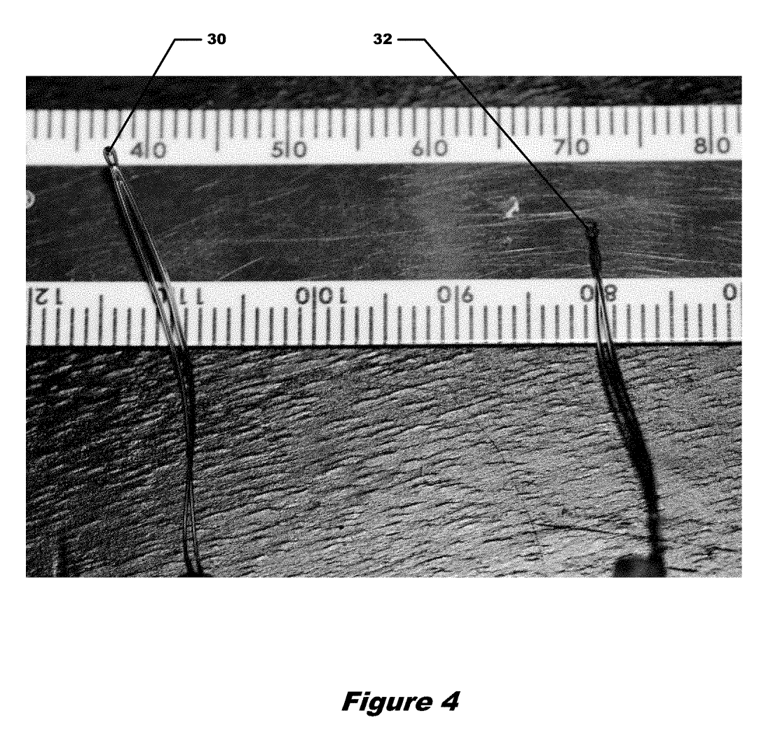

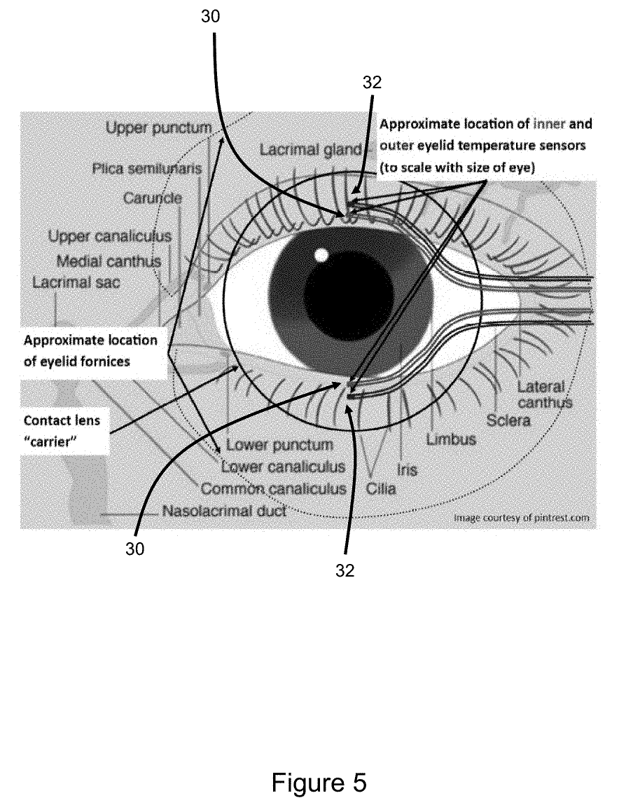

Internal and external eyelid temperature is measured by two sets of Teflon-coated, sealed, waterproof ATH10K1R0 thermistors 30 and 32 as shown in FIGS. 4 and 5. The data from these sensors is processed by a Texas Instruments (Dallas, Tex.) ADS1256 24-Bit, 30 kHz analog-to-digital converter. This information is transferred to the controller.

The controller has a temperature alarm feature, which causes the device to send an alarm signal to the Arduino microcontroller when a pre-selected temperature is reached. This temperature will be set to 60° C. in the ex-vivo testing program—a temperature at which exposures of 5 seconds can cause epidermal damage—and if that alarm temperature is reached, the program will alert the examiner and the program will enter an endless loop in which all power to the heating element will be removed. It is very likely that this alarm state will never be reached, as the temperature sensor samples every 500 milliseconds, and the heating element will be calibrated so that is does not achieve temperatures more than 0.5° C. above target temperature in between samplings.

The heating element 10 will be wrapped in a microfiber cloth 12 for both experiments. The cloth will be dampened with water for the moist heat experiment, and a dry cloth will be used for the dry heat experiments. The cloth will be folded over onto both sides of the heating element, and the temperature sensor 20 will be placed against the cloth, on the outer side of the heating element.

The heating element 10, cloth 12, and temperature sensor 20 will be held against the eyelid with a 100 g beanbag. This will also serve to insulate the temperature sensor from the outside environment, allowing for more accurate temperature readings.

The study heating element device will be heated to 35° C. The device's internal programming will prevent it from exceeding 35° C. (or 95° F., less than body temperature).

Before placing the device on the subject's eyelid, the participant will be reclined in the exam chair and instructed to look upwards. The lower eyelid of EYE1 will be pulled down to expose the inside of the eyelid. The Teflon-coated thermistor 30 sensor will be placed on the inside of the eyelid. The eyelid will then be released. The process will be repeated for the upper eyelid, but the participant will be asked to look downward and toward their nose. See FIG. 5 below for location of eyelid thermistors. They will be placed in these locations to minimize possibility of contact with cornea. This is similar to how filter paper is inserted into the lower eyelid in the Schirmer test procedure. In Schirmer testing, 0.22 mm thick filter paper is folded in the lower lid, commonly in the temporal third of the lid to minimize contact with the cornea. For our purpose we are using thermistors with a 1 mm diameter with 0.3 mm diameter leads. These thermistors are coated in Teflon, so they should be more comfortable, as well as safer, than the use of Schirmer's test strips. Once the eyelid sensors are in place, the participant will be asked to keep both eyes closed throughout the procedure.

At this time, the wrapped study device will be placed on the participant's closed EYE1. A temperature sensor, similar to the eyelid sensors but with its own microcontroller, will be positioned on top of the study device. A bean bag will be placed on top of the sensor and device to insulate the temperature sensor and apply enough weight to bring the device into equal and good contract with the eyelid.

Once the study device, temperature sensors, and beanbag are in place, the program on the microcontroller will be initiated and the device will be warmed to a target temperature of 37° C. over 15 seconds. The temperature will be held at this level for 60 seconds. After this, the temperature of the device will be increased by 1° C. every 75 seconds, allowing 15 seconds for the new temperature to be achieved and 60 seconds at the target temperature. The participant will be constantly monitored throughout the procedure and encouraged to verbally indicate when their level of sensation reaches their interpretation of the 3^{rd} face of the Likert scale.

When the participant indicates the 3^{rd} Likert level, the computer program will be terminated and the device and beanbag will be removed from the participant's eye and the eyelid thermistors removed using the same technique of exposing the inside of the eyelid.

The participant will be able to open their eyes and sit up. Immediately post-treatment, the investigator will perform a Pentacam measurement of the treated eye and will re-examine the anterior segment using the slit lamp.

The participant will be asked to review the Likert scale again before proceeding to treatment with EYE2. EYE2 will use the opposite method from EYE1 (moist treatment if EYE1 received dry treatment, etc.). Post-treatment, EYE2 will be measured again with the Pentacam and examined through a slit lamp.

After both eyes have undergone treatment, the subject will take a break for one hour. After an hour, the participant will again have both eyes examined and Pentacam measurements taken. Treatment will proceed in the same manner as the earlier session, however EYE1 will receive opposite treatment that it did in the earlier testing session (moist treatment if it received dry treatment, etc.). As before, EYE2 will receive the opposite treatment of EYE1. Immediately post-treatment, participants will be given a Pentacam measurement and a slit lamp examination.

The device including the heating elements, temperature sensors and thermistors are used to determine the subjective heat tolerance of the human outer eyelid under both dry and moist conditions. The measurements and data include peak temperature tolerance data, from each trial (moist and dry) will be analyzed to establish: a mean, a median, a standard deviation, a range, skewness, kurtosis, and 95% confidence interval.

The device is also used to determine the correlation of inner and outer eyelid temperatures vis-à-vis warm compress usage. Internal eyelid temperatures will be analyzed to see how long it takes, at a given heating element temperature, to raise internal temperature by 0.25° C. This will be done for dry and moist heat independently. Findings will include a mean, a median, a standard deviation, skewness, kurtosis, 95% confidence interval, best-fit regression and a correlation to best-fit regression.

The device is also used to determine if there are inter-ocular differences in heat tolerance. Peak temperature tolerance data for each eye will be compared for differences between eyes using both dry and moist temperature data together, as well as independently, for each subject. This will be analyzed using a paired t-test to see if the average test subject shows consistent differences between the temperature tolerances of each eye. To determine if there is variation in heat tolerance on different days peak temperature data for each individual, from the two study days, will be analyzed using a paired t-test. To determine if heat tolerance is correlated with skin type peak temperature data for each skin color score will be averaged and plotted graphically. This plot will be analyzed for best-fit regression and for correlation to best-fit regression. To determine if warm compresses cause changes in corneal topography Pentacam data prior to device application and after device application will be analyzed using a paired t-test, examining these five variables: horizontal K value, Vertical K value, Corneal astigmatism, Center corneal thickness, and Peripheral corneal thickness.

As result of the testing and correlation of data, a device for treating Meibomian Gland Dysfunction (MGD) and Dry Eye includes a heating element sized to cover an eye of a person; a cover disposed surrounding the heating element; a temperature sensor disposed on an outside surface of an eyelid of the eye of the person; and a controller in electrical communication with the heating element and the temperature sensor, said controller for setting and maintain a temperature of said heating element.

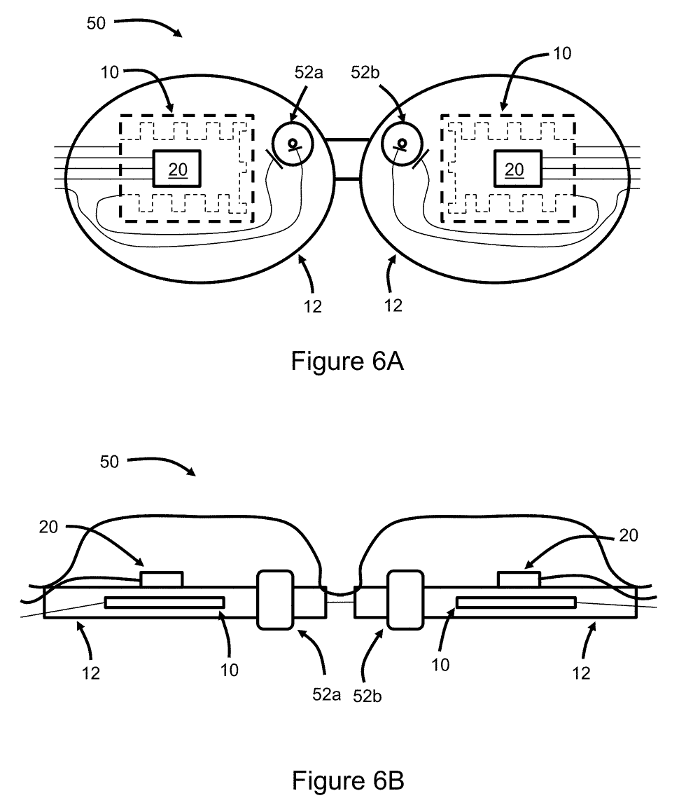

Referring now to FIGS. 6A and 6B, a bilateral device 50 for commercial use is shown. Device 50 includes a temperature sensor 20, a heating element 10 and a flap of material 12 for each eye of a user. Also shown are conductive attachment devices 52a and 52b (e.g., a metallic snap or similar connector) which serves as a safety element by providing a ground from the heating element 10 which prevents heating element 10 activation when the temperature sensor 20 is not in proper contact with the heating element 10. The conductive attachment device and flap of material also provide the benefit of permitting a removable microfiber wrap to be used.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems.

Throughout the entirety of the present disclosure, use of the articles “a” or “an” to modify a noun may be understood to be used for convenience and to include one, or more than one of the modified noun, unless otherwise specifically stated.

Elements, components, modules, and/or parts thereof that are described and/or otherwise portrayed through the figures to communicate with, be associated with, and/or be based on, something else, may be understood to so communicate, be associated with, and or be based on in a direct and/or indirect manner, unless otherwise stipulated herein. Although the methods and systems have been described relative to a specific embodiment thereof, they are not so limited. Obviously, many modifications and variations may become apparent in light of the above teachings. Many additional changes in the details, materials, and arrangement of parts, herein described and illustrated, may be made by those skilled in the art.

Having described preferred embodiments of the invention it will now become apparent to those of ordinary skill in the art that other embodiments incorporating these concepts may be used. Accordingly, it is submitted that that the invention should not be limited to the described embodiments but rather should be limited only by the spirit and scope of the appended claims.

1. An apparatus for treating Meibomian Gland Dysfunction (MGD) and Dry Eye, comprising:
a heating element configured to be disposed proximate to an external surface of an eyelid of a person;
a temperature sensor; and
a controller in electrical communication with said heating element and said temperature sensor, said controller implementing logic that sets a target temperature of said heating element responsive to a temperature measurement obtained from the temperature sensor.

2. The apparatus of claim 1 wherein the temperature sensor is configured to be disposed between the eyelid and eye of the person.

2. The apparatus of claim 2 comprising a second heating element disposed proximate to an external surface of a second eyelid of a second eye of the person, and a second temperature sensor disposed between the second eyelid and second eye, the second temperature sensor being in electrical communication with the controller.

3. The apparatus of claim 2 wherein the eyelid is an upper eyelid, and comprising a second temperature sensor disposed between a lower eyelid of the eye of the person, the second temperature sensor being in electrical communication with the controller.

4. The apparatus of claim 1 wherein the temperature sensor is disposed proximate to the heating element such that the heating element is between the eyelid and the temperature sensor.

5. The apparatus of claim 4 wherein the heating element is disposed in a cloth.

6. The apparatus of claim 1 comprising at least one conductive attachment device that controls activation of the heating element.

7. The apparatus of claim 2 wherein the heating element comprises a Teflon-coated thermistor.

8. The apparatus of claim 1 wherein the logic comprises a temperature alarm feature that generates an alarm signal when a predetermined temperature is detected by the temperature sensor.

9. The apparatus of claim 8 wherein the predetermined temperature is 60° C.

10. The apparatus of claim 1 wherein the target temperature is at least 35° C.

11. The apparatus of claim 1 wherein the target temperature is at least 37° C., and the logic causes the heating element to heat to hold that target temperature for at least 60 seconds.

12. The apparatus of claim 11 wherein the target temperature is increased in increments and held for at least 60 seconds at each increment.

13. A method for treating Meibomian Gland Dysfunction (MGD) and Dry Eye, comprising:
placing a heating element proximate to an external surface of an eyelid of a person; and
with a controller in electrical communication with the heating element and a temperature sensor, setting a target temperature of the heating element responsive to a temperature measurement obtained from the temperature sensor.

14. The method of claim 13 comprising placing the temperature sensor between the eyelid and eye of the person.

15. The method of claim 14 comprising placing a second heating element proximate to an external surface of a second eyelid of a second eye of the person, and placing a second temperature sensor between the second eyelid and second eye, the second temperature sensor being in electrical communication with the controller.

16. The method of claim 14 wherein the eyelid is an upper eyelid, and comprising placing a second temperature sensor between a lower eyelid of the eye of the person, the second temperature sensor being in electrical communication with the controller.

17. The method of claim 13 comprising placing the temperature sensor proximate to the heating element such that the heating element is between the eyelid and the temperature sensor.

18. The method of claim 16 comprising wrapping the heating element in a cloth.

19. The method of claim 13 comprising controlling activation of the heating element with at least one conductive attachment device.

20. The method of claim 13 comprising generating an alarm signal when a predetermined temperature is detected by the temperature sensor.

21. The method of claim 20 comprising setting the predetermined temperature to 60° C.

22. The method of claim 13 comprising causing the heating element to heat to 35° C.

23. The method of claim 13 comprising causing the heating element to heat to a target temperature of at least 37° C., and holding that target temperature for at least 60 seconds.

24. The method of claim 23 comprising increasing the target temperature in increments, and holding the target temperature for at least 60 seconds at each increment.
